SEQUENCE LISTING (8 sequences)

SEQ ID NO: 1 - DNA, 17 bp, synthetic construct
  misc_feature 1..17: note = Synthetic: B-Actin Forward
  source 1..17: mol_type = other DNA
gacctgactgacctcat

SEQ ID NO: 2 - DNA, 20 bp, synthetic construct
  misc_feature 1..20: note = Synthetic: B-Actin Reverse
  source 1..20: mol_type = other DNA
tctccttaatgtcacgcacg

SEQ ID NO: 3 - DNA, 22 bp, synthetic construct
  misc_feature 1..22: note = Synthetic: G6PC Forward
  source 1..22: mol_type = other DNA
acgtgatggtcacatctactct

SEQ ID NO: 4 - DNA, 22 bp, synthetic construct
  misc_feature 1..22: note = Synthetic: G6PC Reverse
  source 1..22: mol_type = other DNA
acattcaagcaccgcaaatctg

SEQ ID NO: 5 - DNA, 20 bp, synthetic construct
  misc_feature 1..20: note = Synthetic: PCK1 Forward
  source 1..20: mol_type = other DNA
acttacatggtgcgaccttt

SEQ ID NO: 6 - DNA, 20 bp, synthetic construct
  misc_feature 1..20: note = Synthetic: PCK1 Reverse
  source 1..20: mol_type = other DNA
atccccaaaacaggcctcag

SEQ ID NO: 7 - DNA, 18 bp, synthetic construct
  misc_feature 1..18: note = Synthetic: HNF4a Forward
  source 1..18: mol_type = other DNA
gcatcttcttttgcgtcg

SEQ ID NO: 8 - DNA, 21 bp, synthetic construct
  misc_feature 1..21: note = Synthetic: HNF4a Reverse
  source 1..21: mol_type = other DNA
tgtaaaccatgtagttgaggt

(19) United States
(12) Patent Application Publication
(10) Pub. No.: US 2023/0265393 A1
(43) Pub. Date: Aug. 24, 2023

(54) COMPOSITION AND METHODS FOR PRODUCING ADULT LIVER ORGANOIDS

(71) Applicant: Rutgers, the State University of New Jersey, New Brunswick, NJ (US)

(72) Inventors: Hatim SABAAWY, Neshanic Station, NJ (US); Fredrick E. WONDISFORD, New Brunswick, NJ (US)

(21) Appl. No.: 17/935,135

(22) Filed: Sep. 26, 2022

Related U.S. Application Data
(60) Provisional application No. 63/248,936, filed on Sep. 27, 2021.

(51) Int. Cl. C12N 5/071

(52) U.S. Cl. CPC C12N 5/0671; C12N 2513/00; C12N 2501/155; C12N 2501/727; C12N 2500/30; C12N 2501/237; C12N 2506/14; C12N 2506/30; C12N 2500/90

(57) ABSTRACT

Liver metabolism studies are limited by the inability to expand primary hepatocytes in vitro while maintaining their metabolic functions. Human hepatic three dimensional organoids have been established for use in these studies, but hepatic organoids from adult donors had impaired expansion. Methods of achieving expansion of adult donor-derived hepatic organoids (HepAOs) and HepG2 cells (HepGOs) from single cells in organoid cultures using combinations of growth factors and small molecules are described, with assessment of expansion dynamics, gluconeogenic and HNF4α expression, and albumin secretion. The invention discloses conditions including limiting A8301 and incorporating FSK and OSM to allow the expansion of HepAOs from adult donors and HepGOs with gluconeogenic competence. These models increase the repertoire of human hepatic cellular tools available for use in liver metabolic assays.

### CROSS-REFERENCE TO RELATED APPLICATION

The present application claims the benefit of priority to U.S. Provisional Application No. 63/248,936, filed 27 Sep. 2021, the contents of which are hereby incorporated by reference in its entirety.

### GOVERNMENT FUNDING SUPPORT

This invention was made with government support under grants no. CA072720, DK063349, and CA226746, awarded by the National Cancer Institute. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 23, 2023 is named “15290-020US1.xml” and is 11,170 bytes in size. The sequence listing contained in this .XML file is part of the specification and is hereby incorporated by reference herein in its entirety.

### BACKGROUND

### 1. Field of the Invention

The invention relates to the field of medicine and medical research. In particular, the specification discloses methods for production and expansion of liver organoids from hepatocytes obtained from adult donors.

### 2. Background of the Invention

Liver disease accounts for about two million deaths annually worldwide. Chronic liver diseases are multifactorial with main causes including viral infections, excess alcohol intake, fatty liver, autoimmune and drug related liver diseases and liver cancer. The liver architecture forming the hepatic lobule is primarily composed of two types of epithelial parenchymal cells: the hepatocytes and biliary epithelial cells (also known as cholangiocytes), and several nonepithelial stromal cell types including macrophages (Kupffer cells), stellate cells, and sinusoidal endothelial cells. Hepatocytes are the main cells responsible for the majority of liver functions.

Based on the starting material and culture conditions, three-dimensional (3D) multi-cellular spherical culture models can be largely classified into four types: spheroid models grown in scaffold-free conditions, spheres obtained by expansion of adult stem-like cells (ASC) in growth factor supplemented serum-free conditions, organotypic slice cultures obtained by slicing of primary tissues, and organoids obtained from ESC, iPSC, ASC, or stem-like tumor cells and grown in scaffold-based serum-free conditions. Multiple laboratories have designed protocols aimed at generating 3D cultures for hepatic cells derived from liver tissues. While the term “sphere” has been used to represent scaffold-based 3D culture, and organoids could be generated in scaffold-free conditions, for clarity, we limit the use of the term ‘sphere’ or ‘spheroid’ to ‘scaffold-free’ 3D cultures, while we reserve the term ‘organoid’ to broadly represent 3D multicellular ‘scaffold-based’ cultures in extracellular matrix (ECM), from single stem-like and/or organoid forming cells. In general, an organoid is a three-dimensional (3D) tissue structure that highly resembles the in vivo organ from which it originated. In this invention, researchers have developed a new method to expand adult donor-derived hepatic organoids (HepAOs) and HepG2 hepatoma cells (HepGOs) from single cells in organoid cultures using combinations of growth factors and small molecules.

Although some have reported the successful isolation, expansion, and differentiation of prenatal liver cells into liver organoids, previous work has suggested that adult liver tissue from humans, but not mice, requires regulation of TGFβ βsignaling and cAMP activity for long-term expansion. Addition of a glycogen synthase kinase 3 (GSK-3) inhibitor (CHIR99021) and keratinocyte growth factor 7 (FGF7), and by removing FSK from the hepatocyte culture media has resulted in some improvement, but these studies demonstrated that mouse and human postnatal hepatic organoids displayed higher expansion capacity than adult organoids. Therefore, adult donor derived human organoids have a limited and/or impaired expansion.

The gluconeogenic pathway has been heavily linked with metabolic and physiological shifts in many liver diseases. However, the effects of hepatic organoid culture conditions on the gluconeogenic function of differentiated hepatic cells in previous organoid studies have not been well-characterized. Hepatoma cells could be expanded in 3D cultures using the hanging drop method and/or with ECM hydrogels for use in drug metabolism assays. Previous studies reported variations of serum-free 3D culture conditions for generating liver organoids. Growth factor supplementation in these conditions were guided by the knowledge of the molecular pathways regulating liver embryonic development, including the FGF, HGF, Wnt, BMP, RA and TGFβ pathways that promote hepatic progenitor proliferation, migration, and survival. Clevers and colleagues established hepatic organoid cultures by modifying their original intestinal organoid methods, which included epithelial cell culture within a 3D laminin- and collagen IV-rich ECM; Matrigel™, together with supplementation with EGF (a mitogen), utilized in seminal hepatic organoid studies, Noggin (a BMP inhibitor), and R-spondin-1 (Rspo1) (an enhancer of WNT signaling and ligand of the stem cell protein LGR5). However, hepatic organoids could be established from human fetal liver cells from donor embryos of 11-20 weeks of gestation, while organoids from adult primary human hepatocytes were limited in their expansion, as indicated by Hu et al. Additional published methods have indicated that viral infection was needed for expansion and production of liver organoids. See Levy et al. Nat. Biotechnol. 33, 1264-1271, 2015. Methods involving viral infection are not preferable because they increase the oncogenic potential of the virally infected cells leading to hepatocyte immortalization, loss of hepatocyte phenotype, and safety concerns in transplant applications.

Therefore, there is a need in the art for methods for generating adult donor-derived hepatic organoids (HepAOs) and hepatoma cells in organoid cultures (HepGOs) from a single cell or single cells.

### SUMMARY OF THE INVENTION

The invention relates to compositions and methods for production and expansion of liver organoids from adult single hepatocytes. In particular, the invention includes methods of making liver organoids and its use. Specifically, embodiments of the invention include an isolated hepatocyte in three-dimensional culture, which is derived from a donor adult hepatic cell or a hepatoma, and which is gluconeogenic.

In another embodiment, the invention comprises a liver organoid comprising at least one hepatocyte of claim 1.
In another embodiment, the invention includes a method of producing a liver organoid in serum-free three-dimensional culture conditions, comprising: (a) obtaining at least one hepatocyte in a single cell suspension; (b) placing the hepatocyte in three-dimensional organoid culture with extracellular matrix and isolation medium (IM) for 4 days; (c) substituting the isolation medium with expansion medium (EM) containing about 1 μM to about 10 μM Y-27632 and optionally further containing one or more of about 1 mM to about 20 mM forskolin, about 1 nM to about 1 μM A83-01, about 1 ng/mL to about 20 ng/mL oncostatin M, and about 1 ng/mL to about 50 ng/mL bone morphogenetic protein 7 (BMP7; (d) culturing the organoid culture for about 1-2 weeks, replenishing the EM about 2-4 times per week; (e) replacing the EM with DM; (f) culturing the organoid culture for about 1 week; (g) replacing the DM with GPM to subject the organoid culture to glucose starvation; (h) culturing the organoid culture for about 24 hours; and (i) optionally measuring glucose secretion by the organoid.
In some preferred embodiments of the method above, the EM contains about 1 mM to about 20 mM forskolin, about 1 ng/mL to about 10 ng/mL oncostatin M, and about 1 ng/mL to about 50 ng/mL bone morphogenetic protein 7. In other preferred embodiments of the method above, the EM contains about 10 mM forskolin and about 10 ng/mL oncostatin M for the first 6 days of culture in EM and about 10 mM forskolin and about 25 ng/mL BMP7 after 6 days of culture. In some embodiments, the hepatocytes are adult primary hepatocytes from an adult donor, preferably an adult human donor. In these embodiments, preferably single hepatocytes are subjected to a first expansion phase involving 3D culture in EM containing 10 mM forskolin and 10 ng/mL oncostatin M for 6 days and starting at day 7 in a second expansion and differentiation phase involving 3D culture in EM containing 10 mM forskolin and 25 ng/mL BMP7 for 8 days. In some embodiments, the hepatocytes are obtained from a hepatoma or hepatoma cell line. In these embodiments, preferably single hepatoma cells are subjected to an expansion and differentiation phase involving 3D culture in EM containing 10 mM forskolin, 10 ng/mL oncostatin M, and 25 ng/mL BMP7 for 8 days. In some preferred embodiments, the hepatoma or hepatoma cell line show significant reductions in AFP tumor marker expression.
In another embodiments, the invention includes a liver organoid produced by the methods discussed above, which is gluconeogenic.

### BRIEF SUMMARY OF THE DRAWINGS

FIG. 1A is a rough schematic drawing of the single cell derived hepatic organoid generation process. FIG. 1B is a set of photographs showing brightfield images of human derived organoids derived from dissociated single cells after expansion for the indicated times. The top of the figure shows conditions Cl through C6 tested on primary hepatocytes and HepG2 cells. FIG. 1C is a graph presenting the percentage organoid forming efficiency from primary hepatocytes in 3D organoid cultures (HepAOs) in different media conditions. FIG. 1D is a set of bright field images of expanded primary hepatocytes in 3D organoid cultures (HepAOs) grown in different media conditions. FIG. 1E is a graph showing the number of HepAOs generated using three distinct small molecule EM conditions. FIG. 1F is a set of images showing hepatic organoids generated in C6 EM expanded for 4 weeks, followed by Differentiation Media (DM) with BMP7 for 2 weeks. The indicated stains are DAPI for nuclei, HNF4α for hepatocytes, and albumin (ALB) for hepatocyte function.

FIG. 2A is a list of 3D organoid EM culture conditions (C1-C6) examined for expansion of donor derived adult hepatocytes. FIG. 2B is a set of photographs showing representative images of HepAOs generated in EM supplemented with Forskolin (F), A83-01, and Oncostatin M (OSM).

FIG. 3A shows the conditions G1 through G6 (various EM with supplemental conditions combined with differentiation in DM for 3D culture of HepG2 hepatoma cells to form HepGOs. FIG. 3B is a graph showing counts of HepGOs derived using BMP7 and A8301 supplemented conditions compared to regular HepG2 growth media. FIG. 3C is a graph showing counts of HepGOs derived using various supplemented conditions as indicated.

FIG. 4A is a set of photographs providing representative images of HepGOs derived under various conditions described in FIG. 3A. FIG. 4B and FIG. 4C are graphs showing the expression of G6PC and PCK1, respectively, in standard EM and EM supplemented with various core molecules as indicated. FIG. 4D and FIG. 4E are graphs showing the expression of G6PC and PCK1, respectively, in EM conditions (C3-C6) that produced the highest number of organoids. FIG. 4F is a graph showing the AFP fold change of expression of HepGOs grown under different conditions as indicated.

FIG. 5A, FIG. 5B, and FIG. 5C are graphs showing the expansion of OCT4 (a surrogate marker for ASC), hepatic marker HNF4a, and gluconeogenic markers G6PC and PCK1.

FIG. 6A is a graph showing the fold change in the expression of PKC1 post induction of differentiation in differentiation media (DM) in HepAOs from three donor groups. FIG. 6B is a set of images showing expression of SOX2 in expanded organoids.

FIG. 7A is a set of photographs showing representative IF images of HepGOs expanded for the indicated times. FIG. 7B and FIG. 7C are graphs showing the quantitation of expression of HNF4α and the functional marker albumin (ALB) in HepGOs at the indicated times. FIG. 7D is a graph showing glucose production in HepGOs expanded in C6 EM.

FIG. 8A is a Z stack confocal image showing expression of ALB as a functional hepatocyte marker, and HNF4α, a marker used to determine hepatocyte fates. FIG. 8B is a set of images of a representative organoid used for volumetric analysis. FIG. 8C is a representative volumetric ALB/DAPI overlay for size estimates of organoids.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0 ±0.2.

As used herein, the term “organoid” refers to a miniaturized and simplified version of an organ, produced in vitro in three dimensions that shows realistic micro-anatomy of the organ, i.e., an artificially grown three-dimensional tissue construct that mimics its corresponding in vivo organ such that it can be used to study aspects of that organ in a tissue culture dish.

As used herein, the term “BM” refers to basal medium, see Table 2.

As used herein, the term “EM” refers to expansion medium, see Table 2.

As used herein, the term “DM” refers to differentiation medium, see Table 2.

As used herein, the term “GPM” refers to glucose production medium, see Table 2.

As used herein, the term “IM” refers to isolation medium, which is made up of EM, but further supplemented with 25 ng/mL recombinant human Noggin (a bone morphogenic protein antagonist).

As used herein, the term “OSM” refers to oncostatin M.

As used herein, the term “FSK” refers to forskolin.

As used herein, the term “HNF4a” refers to hepatocyte nuclear factor 4α, a hepatocyte marker.

As used herein, the term “DAPI” refers to 4′,6-diamidino-2-phenylindole, a fluorescent stain.

As used herein, the term “FGF7” refers to keratinocyte growth factor 7. The term “FGF19” refers to fibroblast growth factor 19.

As used herein, the term “TGFβ” refers to transforming growth factor 3. “A8301” is a TGFβ inhibitor.

As used herein, the term “ALB” refers to albumin.

As used herein, the term “ASC” refers to adult stem-like cells.

As used herein, the term “PCK1” refers to phosphoenolpyruvate carboxykinase 1.

As used herein, the term “G6PC” refers to glucose-6-phosphate catalytic subunit.

As used herein, the term “Y-27632” is a rho-associated protein kinase (ROCK) inhibitor.

As used herein, the term “CHIR99021” is a glycogen synthase kinase 3 (GSK-3) inhibitor.

As used herein, the term “extracellular matrix (ECM)” refers to the non-cellular component present within tissues and organs that provides physical scaffolding for the cells. The main components are protein, glycosaminoglycan, and glycoconjugate. ECM can be obtained commercially, for example as Matrigel™. Other sources and types include natural extracellular matrices including collagen, elastin, laminin, entactin, fibrin, fibronectin, tenascin, thrombospondin, proteoglycans such as agrin, heparan sulfate proteoglycan, neurocan, versican, and fibromodulin, reconstituted with different isoforms, ratios, and in multiple combinations, and synthetic or artificial extracellular matrices formulated from reconstituted natural matrices and including scaffolds such as cultrex, geltrex, synthimax, and others derived from hydrogels, polyacrylamide and polyethylene glycol hydrogels, vitrogels, fibronectin-derived three-amino-acid RGD peptides, and other synthetic or natural and synthetic combinations.

As used herein, the term “gluconeogenesis” refers to a metabolic pathway that results in the generation of glucose from certain non-carbohydrate carbon substrates, including but not limited to lactate, glycerol, amino acids, and the like.

As used herein, the term “gluconeogenic” refers to glucose production from small metabolites with fasting. A cell with “gluconeogenic potential” or which is “gluconeogenic” is a cell that produces glucose from small metabolites with fasting.

As used herein, the term “HepG2 cells” refers to a human hepatoma or hepatocyte carcinoma. These cells can be subjected to 3D culture to produce organoids (HepGOs) with hepatocyte-like function and metabolic competence. The same conditions also allowed the expansion of adult primary liver donor-derived organoids (HepAOs).

As used herein, the term “3D culture” refers to a cell culture environment and process that allows single cells to grow and interact with surrounding extracellular framework in three dimensions. 3D culturing can be done with or without a supporting scaffold. The culturing techniques used in the methods described here involved using extracellular matrix as a scaffold .

As used herein, the term “serum-free” refers to culture conditions without fetal bovine serum and without serum substitutes.

As used herein, the term “donor adult hepatic cell” refers to an adult liver cell obtained from an adult donor.

As used herein, the term “hepatoma” refers to hepatocyte carcinoma.

### 2. Overview

Here, the current knowledge of hepatic organoid 3D cultures was expanded using hepatoma and primary donor derived human hepatocytes to report the effects of different media conditions on key genes associated with the gluconeogenic pathway (phosphoenolpyruvate carboxykinase 1 (PCK1) and glucose-6-phosphate catalytic subunit (G6PC). Multiple organoid culture conditions as previously reported were examined and the effects of oncostatin M (OSM), a member of the IL-6 family of cytokine linked to liver bud formation during embryogenesis, FSK, a diterpene highly efficient at increasing concentrations of cAMP, and the TGFβ inhibitor, A8301, causing downregulation of TGFβ, which correlated with specification of hepatoblast towards the hepatocyte fate were examined. An organoid culture condition was designed in a single culture medium combination that limits A8301 and includes FSK and OSM to allow the expansion of HepG2 cells in 3D cultures into cells with hepatocyte-like function and metabolic competence in HepG2 organoids (HepGOs). This condition also allowed the expansion of adult primary liver donor-derived organoids (HepAOs) from single cells in 3D culture assays.

### 3. Summary of Results

Liver organoids were successfully generated. The tested conditions allowed the generation of HepAOs and HepGOs from single cells in 3D cultures. Expanded HepGOs grown in the most preferred condition maintained detectable gluconeogenic expression in a spatiotemporal distribution for at least 8 weeks. Preferred growth conditions were created by limiting A8301 and incorporating FSK and OSM to allow the expansion of HepAOs from adult donors and HepGOs while maintaining gluconeogenic competence. These models increase the repertoire of human hepatic cellular tools available for use in liver metabolic assays. Thus, in summary, the methods provided here allow the generation of HepAOs and HepGOs from a single cell or suspension of single cells in 3D cultures.

### 4. Embodiments of the Invention

Liver organoids have offered an expanded platforms to study human cells at the 3D level in scaffold-based serum-free defined conditions. Previously, organoids have been derived from tissue resident ASCs, induced pluripotent stem cells, and amniotic cells using different combinations of growth factors and small molecules. Evolving organoid cell-based and cell-free strategies could offer alternatives to liver transplantation for patients with end-stage liver diseases. However, the capacity of different molecules to generate rapidly expanding metabolic competent organoids that could express gluconeogenic target genes has not been discussed in previous studies.

Upon blocking Notch signaling and addition of fibroblast growth factor 19 (FGF19), bone morphogenetic protein 7 (BMP7), and dexamethasone for differentiation, human cells are fated toward a hepatocyte phenotype, suggesting that adult liver tissue from humans, but not mice, requires regulation of TGFβ signaling and cAMP activity for long-term expansion. Further improvements in generation of hepatic organoids from postnatal fetal cells could be accomplished by the addition of a GSK-3 inhibitor (CHIR99021) and FGF7 and removing FSK from the 3D culture media. Nusse and colleagues (Wang, et al., Self-renewing diploid Axin2(+) cells fuel homeostatic renewal of the liver. Nature 524: 180-185, 2015, doi: 10.1038/nature14863) have reported expansion of mouse hepatocytes and demonstrated that mouse and human fetal-derived hepatic organoids displayed higher expansion capacity than adult donor derived human organoids. Different conditions of expansion medium (EM) were examined and conditions that were associated with expansion of organoids and upregulation of gluconeogenic targets were identified. The data suggested that the supplemented molecules act in a concerted manner in order to expand organoids, increase hepatocyte nuclear factor 4α (HNF4α), which regulates hepatocyte proliferation and increases relative gluconeogenic hepatocyte expression, or in an opposing manner to limit these effects. These findings shed some light on the roles these growth factors and small molecules play in expansion rates, proliferative capabilities, HNF4α, G6PC, PCK1 and albumin (ALB) expression. Contrary to prior reports, TGFβ should not be included in the media for expansion or differentiation.

A variety of cells which could contribute to functional liver regeneration have been identified in the fetal and adult liver both under normal and injury conditions. Mature hepatocytes have been demonstrated to display bi-lineage differentiation potential in vivo by transdifferentiating into biliary epithelial cells. However, long-term 3D culture of liver progenitor cells had remained challenging. Evidence suggests that liver progenitor cells are not detectable based on the expression of adult progenitor cell markers such as Lgr5 in the healthy adult liver, but become activated upon injury and could contribute to the remarkable regenerative liver response. A cocktail of three small molecules Y-27632 (a rho-associated protein kinase (ROCK) inhibitor), A8301 (a TGFβ inhibitor), and CHIR99021 (a GSK-3 inhibitor) could convert rat and mouse hepatocytes in vitro into small proliferative bipotent progenitor cells. These cells do not resemble hepatocytes morphologically, yet they retain their proliferative capacity and hepatic differentiation ability. Bipotent epithelial liver organoids could be isolated and expanded from perinatal cells and/or bile ductal fragments and differentiated into mature and functional hepatocytes.

Oncostatin (OSM), a member of the IL-6 cytokine family, has been linked with primary formation of the liver bud during embryogenesis. Two of the organoid formation conditions were supplemented with OSM. The effects of forskolin (FSK, a diterpene), which is highly efficient at increasing concentrations of cAMP via the activation of adenylate cyclase (AC), were examined. While reported protocols for rapidly expanding liver organoids utilized FSK, and FSK was shown to induce proliferation of biliary duct cells in vivo, it was unclear if FSK effects in adult hepatic organoids are by directly interacting with the catalytic subunit of AC. Notwithstanding, FSK appears to be an important compound in the rapid expansion of liver organoids. On the other hand, A8301, a transforming growth factor β (TGFβ) Inhibitor and reported component of the hepatic organoid conditions, has been recognized for its WNT-3a modulatory role and its potential to aid in the maintenance of bipotent hepatoblasts. Downregulation of TGFβ and Notch are highly correlated with specification of hepatoblast towards the hepatocyte fate. However, we demonstrated that A8301 could negatively interact with other factors in the 3D culture media to affect expansion rates, and whether the gluconeogenic potential with glucose-6-phosphate catalytic subunit (G6PC) and phosphoenolpyruvate carboxykinase 1 (PCK1) expression differs after differentiation.

Gluconeogenesis is the de novo production of glucose from endogenous carbon sources. Enhanced hepatic glucose production causes fasting hyperglycemia in diabetic patients. Primary hepatocytes are the current gold standard for in vitro studies of gluconeogenesis. Similar to primary hepatocytes, hepatoma cells can be expanded in 3D cultures as 3D spheroid models for use in drug metabolism and liver function assays. Previous studies suggested that glucose release is mainly compensated for by glycogenolysis in the basal state but is provided by gluconeogenesis in the presence of glucose substrates. Moreover, HepG2 cells in serum-containing spheroid culture demonstrated less glucose metabolism abilities in terms of glucose consumption, intracellular glycogen content, gluconeogenesis rate, and sensitivity to glucose modulator hormones like glucagon.

HepGOs in serum-free defined 3D organoid culture, including extracellular matrix. Were demonstrated to contain cells with hepatocyte-like function and metabolic competence responding to glycerol and hormonal glucagon stimuli, suggesting that HepGOs could be further developed for glucose metabolism studies. Multiple media conditions were examined for generating liver organoids and were assessed for their ability to generate glucose and utilize different substrates for gluconeogenesis.

First, multiple well-differentiated hepatoma cell lines (HepG2, PLC/PRF/5, and HuH7) in 3D culture were considered for their abilities to form serum-free hepatic 3D cultures. Among these cells, the effect of glucose on cell growth, proliferation, and survival was examined. HepG2 cells could proliferate faster and their 3D culture size was smaller than that of HuH7 and PLC/PRF/5 cells. HuH7 and PLC/PRF/5 cells could proliferate in 2D culture but HuH7 became elongated and slowly stopped proliferating, while PLC/PRF/5 cells formed smaller and fewer cultures than HepG2 cells under 3D conditions. When cultured under low glucose (corresponding to normoglycemia (1 g/L)), the proliferation of HepG2 cells was not significantly affected, while the proliferation rate of HuH7 cells was highly reduced in normoglycemic conditions, similar to previous reports, suggesting that HepG2 cells could be utilized to optimize hepatocyte-like and metabolic functions in 3D culture.

Here, increased knowledge was provided on the optimum conditions for generation of liver organoids to support future incorporation of these platforms in the clinical settings for use in liver regeneration, metabolic assays for drug screening, diabetes research, and studies related to gluconeogenesis. Because of the shortage of liver donors and the lack of methods to expand adult hepatocytes while maintaining hepatocyte metabolic functions, these methods fill a previously unmet need by providing a mix of growth factors and small molecules that allow expansion of adult liver cells in 3D organoid cultures which provide a significant and unexpected improvement in both the number of organoids and their gluconeogenic ability. These organoids had a surprising new function therefore can be used for liver replacement in the absence of donor organs.

The methods and conditions that best support hepatic organoid expansion and gluconeogenic expression have been identified. The organoids described here are responsive to glucagon and secrete glucose, responses never shown by others. Thus, the organoids have unique and surprising features. Incorporating both oncostatin and BMP7 in the differentiation protocols and performing it in sufficient time to induce hepatic organoid formation is an important feature in the method of producing these organoids with significant and unexpected improvements in the number of organoids produced and their gluconeogenic ability. Unlike previous organoids made from adult cells, which cannot be maintained in 3D culture for extended times, these organoids can be used in liver transplant, replacing the need for organ donation.

In certain embodiments of the invention, a method of producing organoids that are responsive to glucagon and can secrete glucose is provided. In one preferred embodiment, donor adult hepatocytes are collected from an adult donor and prepared as a single cell suspension in multi-well plates. The donor is a mammal, preferably an adult mammal, and most preferably an adult human. These cells are placed in 3D organoid culture under the following conditions. About 5,000 cells were seeded at a concentration of 100 cells per microliter of the ECM Matrigel™ or Cultrex™, and 50 μL of Matrigel™ including cells were placed in each well of a 24-well culture vessel with 500 μL in isolation medium (IM). This IM contains Expansion Medium (EM) supplemented with 25 ng/mL recombinant human Noggin, 30% (v/v) Wnt-3a conditioned medium, and 10 nM Rho Kinase (ROCK) Inhibitor (Y-27632). After 4 days, the IM is replaced with EM. The EM contains basal medium (BM) further supplemented with 1:50 B27 Supplement (without vitamin A), 1:100 N2 Supplement, 1 mM N-acetylcysteine, 10% (v/v) Rspondin1 conditioned medium, 10 mM Nicotinamide, 10 nM recombinant human [Leu15]-Gastrin-I, 50 ng/mL recombinant human EGF, 100 ng/mL recombinant human fibroblast growth factor 10 (FGF10a), and 25ng/mL recombinant human HGF. EM is supplemented with 10 mM Forskolin (F), and with 10 ng/mL Oncostatin-M (OSM) with OSM kept in the medium only for the first six days of culture.

BM is Advanced DMEM/F12 (ThermoFisher Scientific™ (catalog number 1263401) Dulbecco's Modified Eagle Medium/Ham's F-12, supplemented with 1% penicillin and streptomycin, 1% Glutamax (ThermoFisher Scientific™ (catalog number 35050061) L-alanyl-L-glutamine dipeptide in NaCl), and HEPES 10 mM.

The cells are cultured in EM for about 1-2 weeks, at which time the single cells are forming each at least an organoid composed of at least 50-60 cells and reaching 100 μm in diameter. The EM is replaced with hepatocyte differentiation medium (DM) for about 1 week. DM is made up of BM, further supplemented with 1:50 B27 supplement without Vitamin A, 1:100 N2 supplement, 1 mM N-acetyl cysteine, 10 nM recombinant human [Leu15]-Gastrin I, 50 ng/mL recombinant human EGF (epidermal growth factor), 0.5 μM A8301, 10 μM DAPT (N-[N-(3,5-difluorophenylacetyl)-L-alanyl]-S-phenylglycine t-butyl ester), 3 μM dexamethasone, 25 ng/mL BMP7, and 100 ng/mL recombinant human FGF 19 (fibroblast growth factor 19). During this time, the cells demonstrated hepatocyte like morphology and demonstrated expression of hepatic markers and albumin secretion.

After differentiation, the cells are subjected to glucose starvation for about 24 hours by replacing the medium with glucose production medium (GPM, which is BM supplemented with either 1-5 mM glycerol, 10 nM glucagon, or a combination of both). After glucose starvation for three hours or 24 hours, the cells are ready for glucose production assays in GPM.

The most preferred conditions for producing liver organoids from adult single cell derived hepatocytes (from an adult donor) are C6 conditions with two main phases: expansion and differentiation. Hepatocytes are obtained from the liver of an adult donor and placed in single cell suspension in basal medium. These cells are subjected to an expansion phase in two stages, during which the hepatocytes divide in 3D culture containing extracellular matrix. This phase involves culturing in expansion medium (EM) containing forskolin (about 1 mM to about 100 mM, preferably about 10 mM or about 20 mM) and oncostatin M (about 1 ng/mL to about 100 ng/mL, preferably 10 ng/mL or about 20 ng/mL) for about 3 days to about 15 days or about 2 weeks, preferably about 6 days, and then culturing in expansion medium (EM) containing forskolin (about 1 mM to about 100 mM, preferably about 10 mM or about 20 mM) and BMP7 (about 1 ng/mL to about 1 mg/mL, preferably about 25 ng/mL or about 50 ng/mL) starting at about day 7 for about two days to about 14 days, preferably about 8 days. Once expanded sufficiently, i.e., when single cells each form an organoid composed of at least 50-60 cells and reaching 100 μm in diameter, differentiation is induced by replacing the expansion medium with differentiation medium for the differentiation phase. The expanded cells are cultured for about 14 days, preferably about three days to about 10 days, and most preferably about 6 days to about 8 days in differentiation medium. This phase is then followed by optional glucose starvation for a period of about 2 hours to about 72 hours, preferably about 3 hours to about 24 hours, or about one day, during which the cells are left in glucose production medium. The liver organoids produced in this way expand to an unexpected degree and gain function (are gluconeogenic). Unlike prior liver organoids, they can be used for regeneration of liver in patients, avoiding the need for liver donors.

Hepatoma cells have their own innate expansion capability and are treated with a single main step where expansion and differentiation occur together. The hepatoma cells are obtained in a single cell culture in basal medium and then subjected to expansion/differentiation in 3D organoid culture with extracellular matrix in expansion medium supplemented with about 1 mM to about 100 mM, preferably about 5 mM to about 20 mM and most preferably about 10 mM or about 20 mM Forskolin (F), about 1 ng/mL to about 500 ng/mL, preferably about 10 ng/mL to about 50 ng/mL, and most preferably about 25 ng/mL or about 50 ng/mL BMP7, and about 1 ng/mL to about 100 ng/mL, preferably about 5 ng/mL to about 20 ng/mL, and most preferably about 10 ng/mL or about 20 ng/mL Oncostatin-M (OSM) for the length of the 3D culture (i.e., for a period of about 3 days to about 28 days, preferably about 7 days to about 21 days or until appearance of 3D organoids derived from single cells, validated for metabolic competence. At this time, the liver organoids can be used for drug screening assays and as a treatment for liver cancer. These hepatoma or hepatoma cell line derived liver organoids have significant reductions in α-fetoprotein (AFP) tumor marker expression. These organoids can be used for metabolic functional assays, drug screening, and studies related to inducing differentiation of hepatocellular carcinoma for cancer therapy.

Although the preferred conditions and timing are discussed above, the conditions can be altered according to the convenience of the practitioner, as can be easily appreciated by the skilled artisan.

Multiple media conditions for generating liver organoids were examined and assessed the resulting liver organoids for their ability to generate glucose and use different substrates for gluconeogenesis. After testing tens to hundreds of different combinations of serum-free growth factor and small molecule supplementations (including the FGF, HGF, Wnt, BMP, RA, IL6 and TGFβ pathways that promote hepatic organoid formation), the conditions described here were determined to produce organoid cultures from adult hepatocytes.

Prior studies investigating methods for long-term culture of adult human liver (Huch et al., Long-term culture of genome-stable bipotent stem cells from adult human liver. Cell 2015, 160, 299-312) have indicated that adult human hepatocytes require TGFβ signaling and cAMP activity for long-term expansion. A follow up study from the same group (Hu et al., Long-Term Expansion of Functional Mouse and Human Hepatocytes as 3D Organoids. Cell 2018, 175, 1591-1606) showed the generation of hepatic organoids by adding CHIR99021 and FGF7 and removing FSK from the Huch et al. previously reported condition. The 2018 Hu et al. study use human donor embryos of 11-20 weeks of gestation to produce organoids. There is a known limited availability of these tissues and ethical concerns regarding using human embryos, which are not needed for or used in the present inventive methods. Hu et al. indicated that human primary hepatocytes from adult donors have a limited and/or impaired expansion. The results disclosed here provide a possible explanation for the limited expansion found with older methods with the finding that downregulation of TGFβ with A8301 limited expansion, while removing A8301, improves the expansion. The methods of the invention provide conditions that allow production of organoids in larger numbers and which produce liver organoids from expanded single adult cells obtained from adult subjects, which have gluconeogenic ability.

Unlike with previous methods, and the organoids produced by them, it is shown here that using particular additional factors using an appropriate schedule was able to support hepatocyte maintenance and regeneration, and could generate hepatic organoids with enhanced expansion and more similarity to adult liver tissue. Specifically, oncostatin M, a member of the IL-6 family of cytokines linked to liver bud formation during embryogenesis which has not been described previously. Further, BMP7 also was used during differentiation, which also has not been described previously during expansion. The inhibitor A8301 (which causes downregulation of TGFβ in combination with FSK and has been used in attempts to produce liver organoids) limits the expansion of hepatic cells in 3D cultures into cells with hepatocyte-like function and metabolic competence.

Previous studies also used HepG2 cells in serum-containing spheroid culture and demonstrated fewer glucose metabolism abilities in terms of glucose consumption, intra-cellular glycogen content, gluconeogenesis rate, and sensitivity to glucose modulator hormones like glucagon. The studies here have demonstrated that HepG2 cells in serum-free defined 3D cultures contain cells with hepatocyte-like function and metabolic competence and gluconeogenic ability responding to glycerol and hormonal glucagon stimuli. This could allow the use of these HepG2 organoids in diabetes research, a novel application. Collectively, the studies reported here and the various findings discussed above describe several areas of progress in this art. The liver organoids from adult cells produce here can now be incorporated into platforms for liver transplant and liver and diabetes disease modeling, which was not possible using the prior methods.

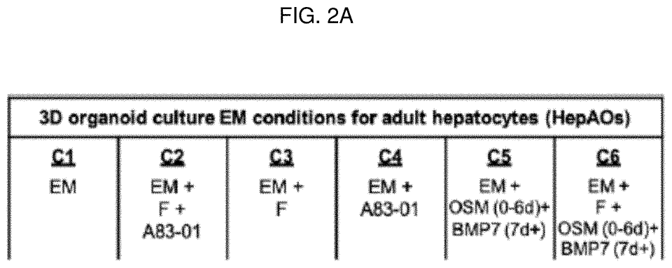

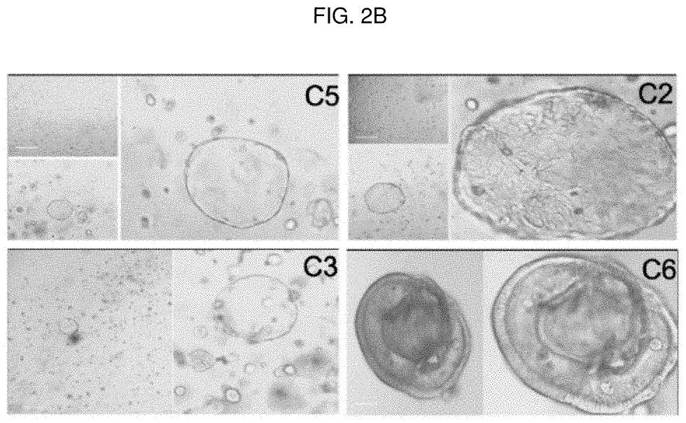

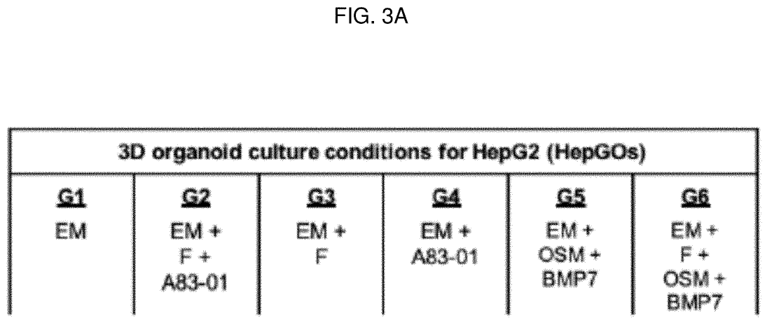

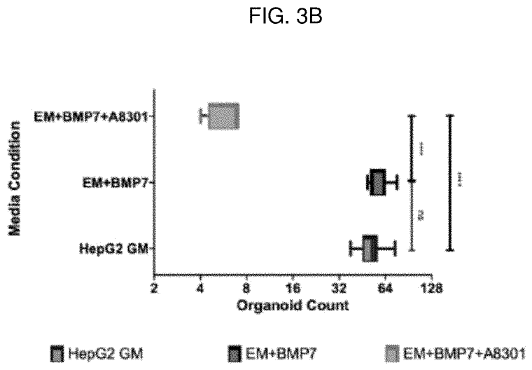

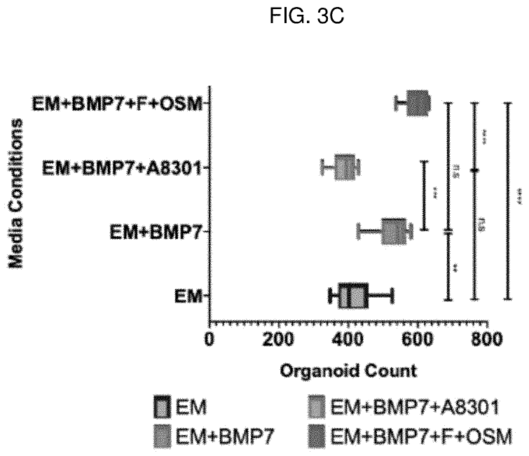

The preferred conditions for expanding cells for this invention are the conditions termed Cl (HepAOs) and G1 (HepGOs) but conditions C2, C3, C4, C5, G2, G3, G4, and G5 also can be used. See FIG. 2A and FIG. 3A. In general, media that contain TGFβ are not used. Thus, for expansion, the expansion medium contains forskolin, and contains oncostatin M for the first few days of expansion (e.g., about 1 to about 14 days, preferably from day 0 to day 6 or the first 6 days of culture), and contains bone morphogenetic protein 7 (BMP7) beginning at about day 7 or when oncostatin M is removed, and continuing until the EM is replaced with DM.

### 5. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Materials and Methods

### A. Cells and Materials

Primary human liver tissues from non-transplantable whole livers or resected healthy liver tissues were obtained from Lonza™. Each tissue is procured in an ethical manner from consenting donors. Tissues were screened for infectious diseases and were negative for HIV, HBV, and HCV. Fresh human hepatocytes in suspension plates with viability <85% were used. Under an IRB approved protocol, the samples were immediately centrifuged at 200g for 5 minutes at 80° C. The supernatant was discarded and 15 mL of basal medium were added to resuspend the hepatocyte cell pellet. The centrifugation procedure was repeated twice with basal medium. The pellet was resuspended using 15 mL of basal medium and the cells were counted using a hemocytometer.

### B. Hepatic Organoid Culture

Human hepatic cells were counted and seeded at a concentration of 100 cells per μL of the ECM Matrigel™ (Corning™). The cell-ECM mix were seeded on 24 well plates Greiner™) that had been incubated 24 hours prior at 37° C. A total of 50 μL of Matrigel™ were added to the center of each well to form a dome shaped chamber and incubated for 5-8 minutes at 37° C. before adding 500 μμL of isolation medium to each well. See Table 1, below. Media specifications and storage times are indicated.

TABLE 1
Overview of the Media used for Generation
of Wnt3a and R-Spondin-1 Factors.

| Medium | | Storage |
|---|---|---|
| Name | Components | Time |
| Wnt-3a Growth | DMEM supplemented with 10% FBS, 1% | 4° C. for |
| Media | Penicillin/streptomycin, and 2% G418 | one week |
| Wnt-3a Harvest | DMEM supplemented with 10% FBS, 1% | 4° C. for |
| Media | Penicillin/streptomycin | one month |
| R-Spondin-1 | DMEM supplemented with 10% FBS, 1% | 4° C. for |
| Growth Media | Penicillin/streptomycin, and 200-300 ug/mL | one month |
| | zeocin | |
| R-Spondin-1 | Advanced DMEM/F-12 supplemented with | 4° C. for |
| Harvest Media | 1% Penicillin and Streptomycin, 1% | one month |
| | Glutamax ™, and 10 mM HEPES | |

After 4 days, the isolation medium was substituted for expansion medium (EM) containing 10 μM ROCK inhibitor (Y-27632). See Table 2 below. The EM was replenished three times per week.

After the first 6 days in culture, the ROCK inhibitor was removed from the media. The cells within the ECM mix were expanded for 14 days to form organoids. After 14 days, the media was supplemented with 25 ng/mL of BMP7, and the organoids were cultured for 5 days before replacing the media with hepatocyte differentiation medium (DM). See Table 2, below. The DM were replenished every 3 days. At day 7 of differentiation, the organoids were used for analyses.

TABLE 2
Overview of the expansion media (EM), Differentiation media (DM)
and other media conditions used for 3D hepatic organoid culture.

| Medium | | Storage |
|---|---|---|
| Name | Components | Time |
| Basal | Advance DMEM/F-12 supplemented with | 4° C. for |
| Medium | 1% Penicillin and | one month |
| (BM) | Streptomycin, 1% Glutamax, and HEPES | |
| | 10 mM. | |
| Human | Basal Medium supplemented with 1:50 B27 | 4° C. for |
| Liver | Supplement (without vitamin A), 1:100 N2 | two weeks |
| Expansion | Supplement, 1 mM N-acetylcysteine, 10% | |
| Medium | (v/v) Rspondin1 conditioned medium, 10 mM | |
| (EM) | Nicotinamide, 10 nM recombinant human | |
| | [Leu15]- Gastrin-I, 50 ng/mL recombinant | |
| | human EGF, 100 ng/mL recombinant human | |
| | FGF10, and 25 ng/mL recombinant human | |
| | HGF. | |
| Human | Liver Expansion Medium supplemented with | 4° C. for |
| Liver | 25 ng/mL recombinant human Noggin, 30% | two weeks |
| Isolation | (v/v) Wnt-3a conditioned medium, and 10 | |
| Medium | nM Rho Kinase (ROCK) Inhibitor (Y- | |
| (IM) | 27632). | |
| Human | Basal Medium supplemented with 1:50 B27 | 4° C. for |
| Hepatocyte | Supplement (without vitamin A), 1:100 N2 | two weeks |
| Differ- | Supplement, 1 mM N-acetylcysteine, 10 nM | |
| entiation | recombinant human [Leu15]-Gastrin I, 50 | |
| Medium | ng/mL recombinant human EGF, 0.5 μM | |
| (DM) | A8301, 10 μM DAPT, 3 μM Dexamethasone, | |
| | 25 ng/mL BMP7, and 100 ng/mL | |
| | recombinant human FGF19. | |
| Glucose | Basal Medium (DMEM) (Gibco ™) | 4° C. for |
| Production | containing no glucose or pyruvate but | 24 hours |
| Medium | supplemented with L-glutamine (2 mM), | |
| (GPM) | HEPES (1.76 g), and penicillin/streptomycin | |
| | (5 ml). BM is supplemented with either 5 | |
| | mM glycerol, 10 nM glucagon, or a | |
| | combination of both | |

### C. Generation and Harvesting of Wnt-3a Conditioned Media

Wnt-3a expressing cells were purchased from ATCC (CRL-2647). Cells were cultured in Greiner Bio-One CELLCOAT™ tissue culture treated T-75 flasks at an initial concentration of 1.5×10⁶ cells. The cells were cultured in 20 mL of Wnt-3a Growth Medium (WGM) and passaged once until 75% confluency was reached. For selection, 0.4 mg/ml G-418 was used, and cells were kept growing on selection medium for a week before producing conditioned medium. The cells were lifted using 3 mL of TrypLE (Life Technologies™) and passaged. The process was repeated until approximately 40-50 T75 flasks were generated. The media were replaced with 100 mL of Harvest Medium (HM). The cells were then incubated in HM for one week without media changes. After one week, the media were collected, and tubes were centrifuged at 500× g for 5 minutes at 8° C. Following this step, the media were filtered using filter cups (Stericup Quick Release, Millipore™). Lastly, 25 mL of the collected harvested medium were separated into 50 mL canonical tubes and used fresh or stored for no more than 2 weeks at 4° C. Recombinant human Wnt3a (R&D, 5036-WN010) was used at a final concentration of 100 ng/mL as a positive control, and Wnt activity of conditioned medium could be examined by the TOP/FOP assay as described.

### D. Plating and Generation of R-Spondin-1 Conditioned Media

R-Spondin-1-expressing 293T cells were purchased (EMD Millipore™, SCC111) and cultured in Greiner Bio-One CELLCOAT™ tissue culture treated T-75 Flasks at an initial concentration of 1.5×10⁶ cells. The cells were cultured in 20 mL of R-Spondin-1 Growth Medium (RGM) and passaged once until 75% confluency was reached using TrypLE (Life Technologies™). The media was changed with fresh RGM every 2 days. To harvest the R-Spondin-1 conditioned media, once cells reached 70% confluency, the media was replaced with 100 mL of HM. The cells were then incubated in this medium for one week without media changes. After this week, the media were collected in 50 mL canonical centrifuge tubes. The tubes were centrifuged at 500× g for 5 minutes at 8° C. Following this step, the media were filtered using 500 mL filter cups (Millipore™), and harvested media were aliquoted into 15 mL canonical tubes and used fresh within two weeks or stored at −20° C.

### E. 3D Hepatic Organoid Culture

Well-differentiated hepatoma cell lines HepG2, PLC/PRF/5, and HuH7 were maintained as previously described. In initial studies, classical culture media was used, containing high glucose concentration (4.5 g/L) corresponding to systemic hyperglycemia. Low glucose concentrations corresponding to normoglycemia (1 g/L) were utilized in parallel to study the effects on cell proliferation. HepG2 cells were first cultured in monolayer. Once 80-90% confluence was reached the cells were collected and used to start the organoid culture. HepGOs were generated on 48 well tissue culture plates (Grenier™). The plates were left overnight at 37° C. before using them to plate the Matrigel™ matrix. Cells were plated at a concentration of approximately 40 cells per μL of Matrigel™. We used a 25 μL drop/well to form the organoid dome at the center and the organoid dome were overlaid with 250 μLs of human expansion medium (EM) with the multiple EM variations tested. The plates were then incubated for 7 days under standard tissue culture conditions (37° C. and 5% CO2). Media was changed every other day. After 7 days of expansion, the media was replaced with differentiation media (DM). The organoids were cultured for an additional 7 days, at which point the organoids were used for analysis.

### F. Recovery of Organoids from 3D Culture

The organoids were removed from the Matrigel™ for use in analyses. To accomplish this, hepatocyte DM were removed from the wells using a 200-microliter pipette. The plate was placed on ice, using an unfiltered 1,000 μL cold pipette tip, and 500 μL of ice-cold PBS buffer was transferred into each well. The PBS was allowed to remain in the wells for 3-5 minutes before collecting it into ice cold 15 mL tubes (placed on ice 30 minutes before starting the procedure). The tubes containing the organoids were then centrifuged at 1,200 rpm for 4 minutes at° C. 4™. The supernatant was removed leaving the organoid pellet intact. Next, 500 μL of cold cell recovery solution was added to the pellet. The pellet was dissolved in the solution by gentle pipetting up and down slowly multiple times. The solutions were then transferred to 1.5 mL Eppendorf™ tubes and left on ice for one hour. The tubes were gently vortexed every 20 minutes. After one hour, the tubes were centrifuged at 4,000 rpms for 5 minutes, and the supernatants were discarded. The pellets were washed twice using 1 mL of ice-cold PBS with centrifugation, and recovered organoid cells were used for different assays.

### G. Assessment of Expression of Hepatic and Gluconeogenic Targets in Organoids

RNA was first extracted from the differentiated organoids using Trizol™. RNA was used for cDNA synthesis using the iScript Kit (BioRad™). Quantitative PCR (Q-PCR) was performed for targets of interest including G6PC, PCK1, and AFP using Bio-Rad™ CFX96. Bio Rad™'s SYBR Green (SG), a asymmetrical cyanine dye used as a nucleic acid stain in Q-PCR super mix was used with the target-specific primers. See Table 3, below for a description of the primers used for hepatocyte expression. The data was analyzed using Excel™ and Prism 8™.

TABLE 3
List of SYBR green Q-PCR primers.

| Target | Forward/Reverse | Sequence | SEQ ID NO |
|---|---|---|---|
| B-Actin | Forward | GAC CTG ACT GAC CTC AT | 1 |
| | Reverse | TCT CCT TAA TGT CAC GCA CG | 2 |
| G6PC | Forward | ACG TGA TGG TCA CAT CTA CTC T | 3 |
| | Reverse | ACA TTC AAG CAC CGCA AAT CTG | 4 |
| PCK1 | Forward | ACT TAC ATG GTG CGA CCT TT | 5 |
| | Reverse | ATC CCC AAA ACA GGC CTC AG | 6 |
| HNF4a | Forward | GCA TCT TCT TTT GCG TCG | 7 |
| | Reverse | TGT AAA CCA TGT AGT TGA GGT | 8 |

### H. Immunofluorescence Staining and Imaging of Hepatic Organoids

IF and sample processing were performed generally as previously described. Prior to being fixed, the organoids were recovered from the Matrigel™ After washing three times with PBS. The organoids were then fixed using 4% PFA and incubated at 37° C. for 15 minutes. The organoids were allowed to precipitate to the bottom of the plate before carefully removing the fixative. The samples were washed three times with PBS and stored at 4° C. before the permeabilization step was done. For permeabilization, the PBS was removed and replaced with 500 μL PBS-0.5% Triton™ X-100. The organoids were then incubated in the PBS-Triton™ X-100 solution overnight at 4° C. The following day, the solution was removed and the organoids were washed three times with PBS. The treated organoids were used immediately for immunostaining for albumin and HNF4α hepatocytic markers. 4′,6-diamidino-2-phenylindole (DAPI) fluorescent stain was used to stain nuclei at a 1:200 dilution, human albumin-specific and human HNF4a antibodies that do not cross react with murine or bovine products were used at 1:200 and 1:100 dilution, respectively. See Table 4, below, for a list and description of the antibodies used for IF assays on organoids. Whole mount staining was performed and the organoids were imaged using a Nikon™ A112 Si Confocal microscope.

TABLE 4
Antibodies

| Manufacturer | AB Type | Target | Species | Dilution | Reference |
|---|---|---|---|---|---|
| Bethyl Labs ™ | Primary | Albumin | Goat | 1:200 | A80-229A |
| Invitrogen ™ | Primary | HNF4α | Mouse | 1:100 | 417700 |
| | DAPI | Nucleic | | 1:200 | |
| | | Stain | | | |
| Invitrogen ™ | Secondary | Goat | Donkey | 2 μg/mL | A11057 |
| Invitrogen ™ | Secondary | Mouse | Donkey | 1:2000 | A21202 |

### I. Confocal Microscopy-Based Volumetric Measurements of Organoids

Confocal scanning microscopy-based volume measurements were done by demarcating cytosolic albumin and nuclear DAPI. Samples were scanned at multiple vertical z-steps to determine the distance between the bottom and the top of the cell at every xy position using water-immersion objectives to minimize the axial mismatch Model. Volume calculation on segmented images was done using ImageJ™plugins Volumes (lepo.it.da.ut.ee/˜markkom/volumest/) and Volume Calculator (im-224agej.net/Volume_Calculator).

### J. Glucose Production Assays

Prior to each glucose production assay, organoid cells were serum starved for 3 and 24 hours. The assay was done in 3D organoid culture media, containing no glucose or pyruvate. For substrate glucose production assays, the glucose production basal media (BM) were supplemented with either glycerol, glucagon, or a combination of both. Data were obtained by obtaining media from wells that contained 12-14 Matrigel™ droplets containing approximately 1,400 organoids. Glucose measurements were done enzymatically with a Glucose Assay Kit (Abcam™).

### K. Statistical Analysis

Statistical analyses were performed using GraphPad™ Prism 8 (GraphPad™ Software Inc., graphpad.com). Data are presented as mean±standard deviation (SD). Statistical significance was determined by t-test and ANOVA, with Dunnet's post-test when appropriate. A P-value <0.05 is represented by a single asterisk; a P-value <0.01 is represented by a double asterisk; and three asterisks indicate P value <0.001; while four asterisks indicate P-value <0.0001.

### Example 2. Expansion Rates and Morphological Changes in 3D Culture Conditions

The Different sets of EM conditions were tested for their potential to generate HepG2 modified hepatic-like organoids (HepGOs). In parallel, freshly isolated human cells were grown in isolation media in order to select and enrich for the ASC fraction harboring the hepatic organoid forming cells.

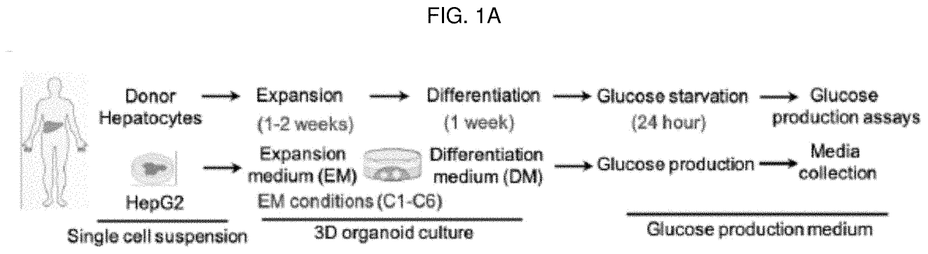

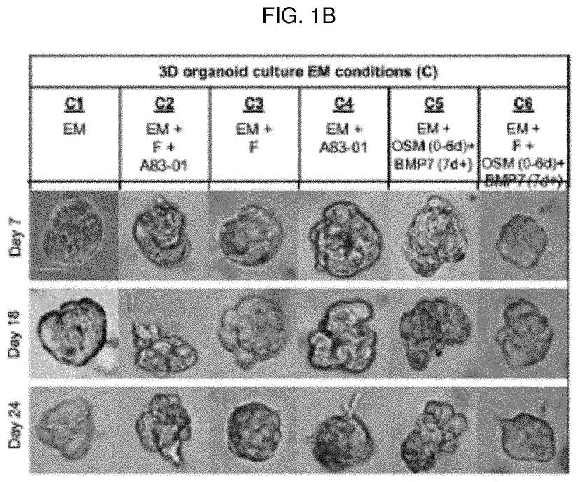

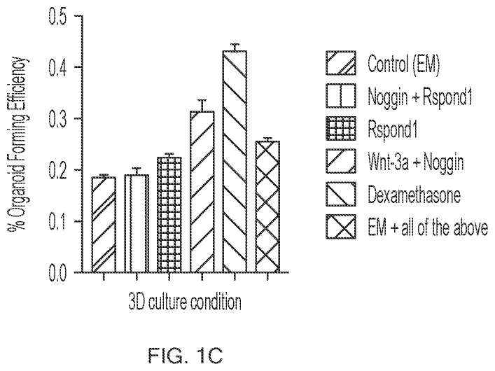

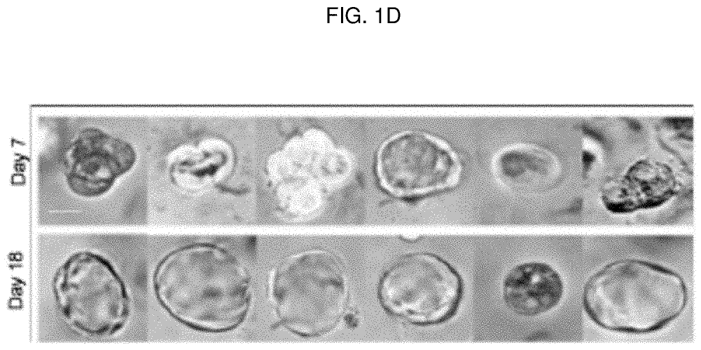

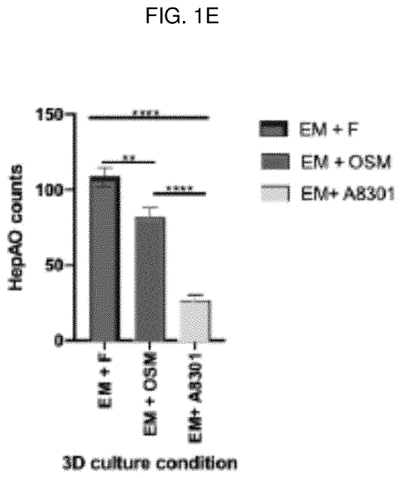

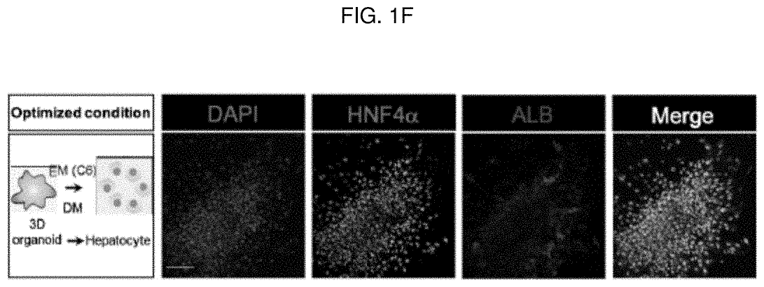

Single cell suspensions were used in 3D organoid culture in various conditions, which were tested for the ability to support the growth and the expansion rates of organoids in Expansion Media (EM) alone (Condition C1) or supplemented with different growth factors/small molecules (Conditions C2-C6). See FIG. 1B.

During the first week of culture, small organoids emerged from Matrigel™-embedded hepatocytes in all culture conditions (FIG. 2A). The organoids expanded from a diameter of 100 mm in one week to a diameter of 400-500 mm within 4 weeks and could be passaged by mechanical dissociation at a ratio of 1:3 every 7-10 days. This allowed refinement of the conditions, eventually resulting in robust organoid growth (see FIG. 2A and FIG. 3B). In 3D organoid cultures from both HepG2 cells (HepGOs) (FIG. 1B and FIG. 2A) and adult donor-derived primary hepatocytes (HepAOs) (FIG. 1D and FIG. 2B), morphological evolution was observed throughout the organoid development process. Different molecules appeared to facilitate the expansion of projections in the HepGOs around day 24 (FIG. 1B) and the repopulation of the lumen in the differentiated HepAOs (FIG. 1D and FIG. 2A).

FIG. 2 (different expansion media (EM) conditions and examples of hepatic organoids) shows in FIG. 2A a list of 3D organoid EM culture conditions examined for expansion of donor derived hepatocytes. FIG. 2B contains representative images of HepAOs generated in EM supplemented with Forskolin (F), A83-01, and Oncostatin M (OSM). Note that not all plated cells expand into organoids, supporting the reported organoid forming potential from ADC and/or organoid forming cells. The scale bar is 100 mm.

FIG. 1A is a rough schematic showing the general process of hepatic organoid generation and testing conditions. Adult donor derived hepatocytes and/or HepG2 cells were obtained in single cell suspension and subjected to 3D culture, expansion in expansion media (EM) under various supplemental conditions, and differentiation in hepatocyte differentiation media (DM), followed by molecular assays for hepatocyte specific markers and glucose production assays for gluconeogenesis.

We assessed the organoid-forming efficiency at one week in single cell derived organoids (FIG. 1B and FIG. 1C). FIG. 1B shows, in the top section, various EM conditions (indicated as C1-C6) tested on primary hepatocytes and HepG2 cells. Column C6 in FIG. 1B lists a preferred condition for organoid growth/primary hepatocytes.

The bottom section of FIG. 1B shows brightfield images of human derived organoids derived from dissociated single cells after expansion for the indicated times under those conditions as indicated. There was no significant difference in the organoid forming efficiency when changing the core EM component, with a trend towards increase in organoid forming efficiency with addition of dexamethasone, an effect that was revered when all core media components were included (FIG. 1C). The percentage organoid forming efficiency from primary hepatocytes in 3D organoid cultures (HepAOs) in different media conditions is shown in FIG. 1C. Bright field images of expanded primary hepatocytes in 3D organoid cultures (HepAOs) grown in different media conditions are shown in FIG. 1D. Experiments were with hepatocytes derived from three different donors. When examining 3D cultured derived from single hepatocyte cell suspensions from adult donors, while previously reported conditions (C1-C3) resulted in similar expansion rates, conditions including F and OSM supplemented media (C5-C6) expanded rapidly over a period of 2 weeks, while conditions including A8301 supplemented media (C4) showed the least number of organoids being formed over the same period of time. See also FIG. 1E, which shows the number of HepAOs generated using three distinct small molecule EM conditions.

Hepatic organoids were generated in C6 EM expanded for 4 weeks, followed by Differentiation Media (DM) with BMP7 for 2 weeks. Organoids were passaged twice followed by IF, staining with DAPI for nuclei, HNF4α for hepatocytes, and albumin (ALB) for hepatocyte function. Since serum-free EM condition C6 (EM+F+OSM) resulted in 3D organoid culture with an intricate morphology for both HepGOs and HepAOs upon long-term expansion (see FIG. 2B), we examined and confirmed the expression of hepatocyte specific HNF4a and albumin (ALB) secretion upon induction of differentiation in these 3D organoids using Immunofluorescence (IF) assays (FIG. 1F). All experiments were performed in triplicate unless otherwise stated. The scale bar is 100 μm. Comparison of counts and conditions were determined by two-way ANOVA with Bonferroni post-hoc test (****p<0.0001, ***p<0.001, **p<0.01). Therefore, the specified conditions resulted in the formation of single cell derived hepatic organoids that could be expanded for more than 8 weeks. These hepatic organoids demonstrated functional hepatocyte features including the expression of hepatocyte specific markers and human albumin section.

### Example 3. Expansion of 3D Organoid Culture With EM and DM in HepGOs

BMP7 accelerates hepatocyte proliferation, and the addition of BMP7 to the EM prior to the start of hepatocyte differentiation accelerated the formation hepatocytes from primary liver tissues, therefore, we assessed the effects of adding BMP7 to HEPG2 organoid 3D culture conditions. BMP7 slightly but not significantly improved HepGO number when compared to growing HepG2 cells in 3D culture using serum-containing growth media (HepG2 GM). See FIG. 3A and FIG. 3B). FIG. 3A lists various culture conditions containing EM with the indicated supplements, combined with differentiation in DM, for 3D culture of HepG2 cells to form HepGOs. In the absence of A8301 (EM+BMP7), HepG2 cells proliferated in 3D organoid cultures at a rate comparable to that of serum-containing 3D culture growth media (HepG2 GM) (FIG. 2A). A key difference between conditions C6 and G6 is the timing of when the various components were added. In C6, OSM is included only during the first 6 days and BMP7 is added after day 7.

On the other hand, conditions including the TGFβ inhibitor A8301 significantly limited the 3D culture and expansion of HepG2 cells. See FIG. 3B, which provides the counts of HepGOs derived using BMP7 and A8301 supplemented conditions, compared to regular HepG2 growth media (GM). This could be in part due to the ability of A8301 to limit Wnt-3a/b-catenin induced cellular proliferation, or the context-dependent coregulation between TGFβ and BMP signaling.

The EM condition C6 (EM+F+OSM+BMP7) also supported higher expansion of HepGOs when compared to EM only or EM+BMP7. See FIG. 3C, which provides the counts of HepGOs organoids derived using various supplemented conditions. The number of organoids grown in conjunction with OSM and Forskolin is the highest when compared to the other conditions. Comparison of counts and conditions were determined by two-way ANOVA with Bonferroni post-hoc test (****p<0.0001, ***p<0.001, **p<0.01, ns, not significant). Therefore, the specified conditions resulted in the formation of single hepatoma cell derived organoids from HepG2 cells that could be expanded for at least 8-12 weeks.

### Example 4. Expression of gluconeogenic G6PC and PCK1 in organoids.

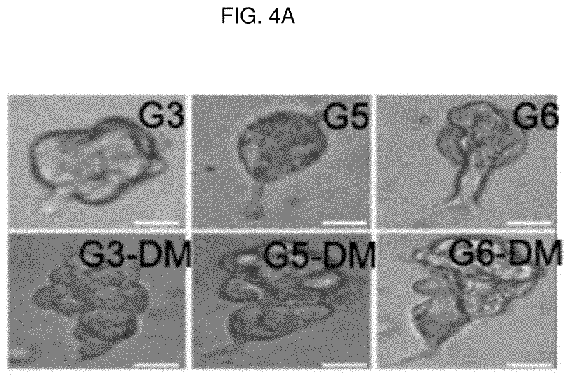

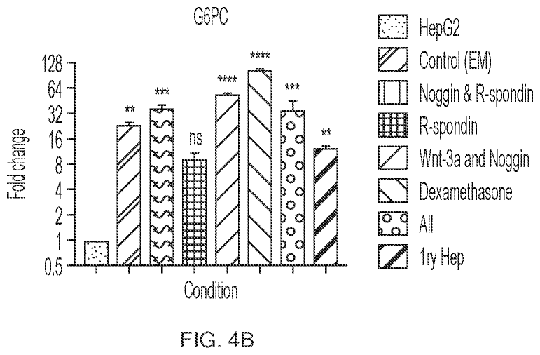

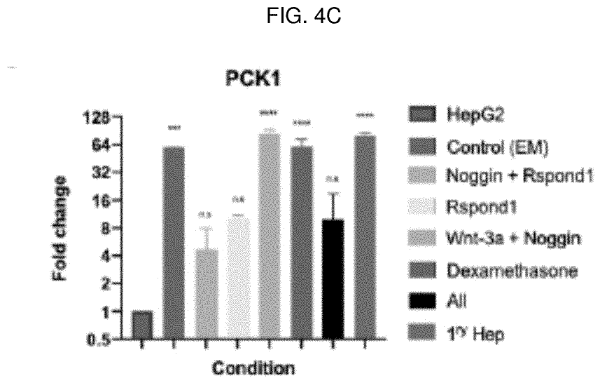

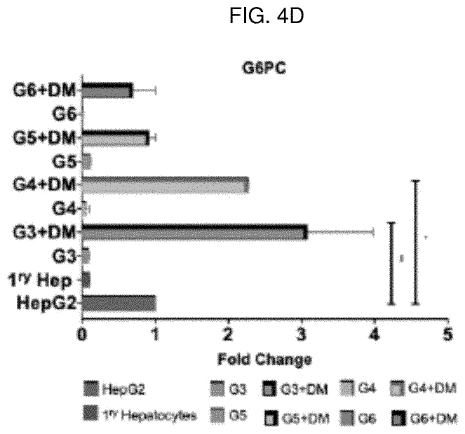

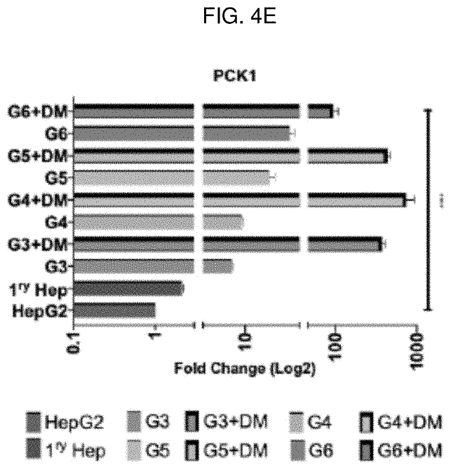

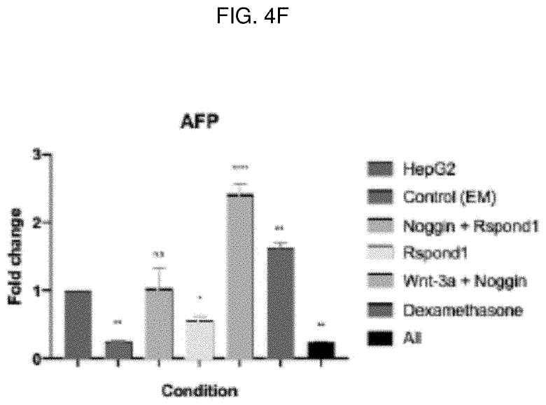

Glucose-6-phosphatase (G6PC), and phosphoenolpyruvate carboxykinase-1 (PCK1) are key enzyme checkpoint regulators of gluconeogenesis, and differences in their expression levels have been linked to type 2 diabetes. Compared to HepG2 GM alone, we first assessed the effects of the core EM components. The overall expression of G6PC and PCK1 were significantly increased (approximately about 8- to 64-fold) when HepGOs were cultured in 3D organoid EM (EM) compared to HepG2 GM (see FIG. 4). We next assessed the effects of the supplements to the EM forming the various EM C1-C6 conditions including variations of (EM+F+OSM+BMP7) on G6PC and PCK1 expression upon expansion for one month and prior to and after induction of hepatocyte differentiation (See FIG. 4).

FIG. 4 shows data on the gluconeogenic marker expression in 3D organoid cultures under different conditions. FIG. 4A shows representative images of HepGOs derived under various conditions described in FIG. 3A. HepGOs were expanded for one month, and then media were supplemented with 25 ng/mL BMP7 for 5 days before replacing the media with hepatocyte DM for one week. The data are from experiments that were done in at least in duplicate with three replicates per condition and were normalized to the endogenous control (β-Actin) and HepG2 GM cultures. The scale bar is 100 μm.

FIG. 4B and FIG. 4C present data on the expression of G6PC and PCK1 in standard EM and EM supplemented with various core molecules. For G6PC, EM supplemented with either a combination of Noggin and Rspon1, Wnt-3a and Noggin, dexamethasone, or all four (EM+Noggin, Rspon1, Wnt3a, and dexamethasone) resulted in a significant increase in the relative G6PC expression (see FIG. 4B). On the other hand, for PCK1, EM supplemented with either Wnt-3a and Noggin, or dexamethasone resulted in a significant increase in the relative PCK1 expression (see FIG. 4C). Interestingly, the addition of all of the tested factors and molecules caused a significant decrease in expression of both G6PC and PCK1, therefore suggesting that some of these factors may play either direct or indirect modulatory roles in the expression of G6PC and PCK1. Notably, EM supplemented with either Wnt-3a and Noggin, or dexamethasone a resulted in a significant increase in expression of both G6PC and PCK1, with PCK1 reaching the level of primary hepatocytes. See FIG. 4B and FIG. 4C.

FIG. 4D and FIG. 4E present data on the expression of G6PC and PCK1 in the EM conditions (C3-C6) that produced the highest number of organoids. The conditions (F+BMP7) and (A8301+BMP7) resulted in induction of G6PC expression after differentiation (see FIG. 4D), while various conditions (EM+F+OSM+BMP7) induced PCK1 expression prior to differentiation and more after differentiation (see FIG. 4E).

With our findings that EM conditions upregulate the expression of G6PC and PCK1 to nearly that of primary hepatocytes, we assessed the expression of hepatocellular carcinomas (HCC) biomarker alpha fetal protein (AFP). FIG. 4F shows the AFP fold change of expression of HepGOs grown under different conditions. (****p<0.0001, ***p<0.001, **p<0.01, ns, not significant). HepGOs that were expanded in 3D EM supplemented with either Wnt-3a and Noggin showed a significant increase in the relative expression of AFP (see FIG. 4F).

Intriguingly, HepGOs which were expanded in either EM alone, EM with Rspnd1, or with all four (EM+Noggin, Rspon1, Wnt3a, and dexamethasone) showed significant reductions in AFP expression (see FIG. 4F).

Altogether, these findings suggest that EM core components and/or some of the supplemented molecules could be interacting with pathways linked to the transcription of AFP and hepatocyte maturation. While initially intriguing, understanding the effects of these molecules on AFP might facilitate future development of new differentiation therapies for HCCs.

### Example 5. Expansion and Freezing of 3D Cultured Hepatic Organoids

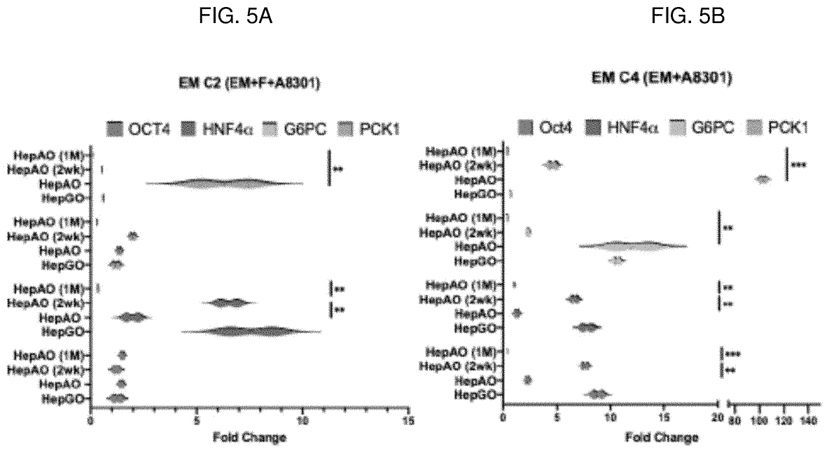

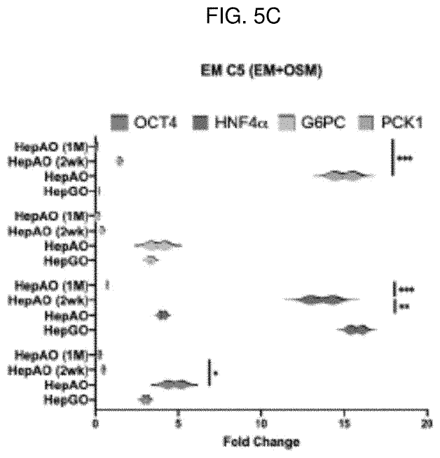

We next assessed the effects of the tested key growth factors and small molecules in supplemented EM conditions on the expression of hepatocyte gluconeogenic target genes upon expansion of fresh or frozen HepAOs and HepGOs. Expression was assessed in HepG2 cells (HepG2), freshly isolated donor derived human liver primary cell suspension (HPLC), two-week frozen and re-expanded HepAOs (HepAO-2wk), and one-month frozen and re-expanded HepAOs (HepAO-1M) (see FIG. 5).

FIG. 5 presents data on expansion and freezing of human hepatic organoids in 3D culture. In particular, expression of OCT4 (surrogate marker for ASC), hepatic marker HNF4α, and gluconeogenic markers G6PC and PCK1 in HepGOs, HepAOs (immediately after EM), and HepAOs after 2-week (HepAO-2wk) or one month (HepAOs-1M) of organoid freezing and thawing under various EM conditions with F+A8301, A8301, and OSM (C3-05) are shown in FIG. 5A, FIG. 5B, and FIG. 5C. The data demonstrate the normalized fold change over base line expression in organoid cultures. Data are from experiments that were done in at least in duplicate with (N=3) donor hepatocytes and multiple HepG2 organoid cultures, each in three replicates per condition.

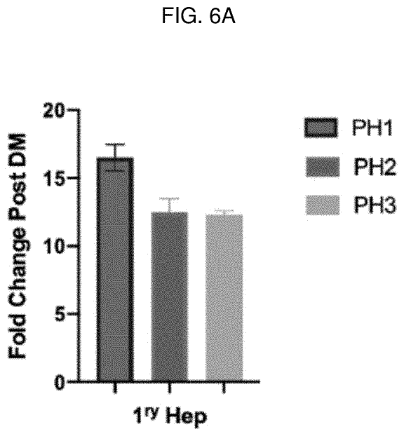

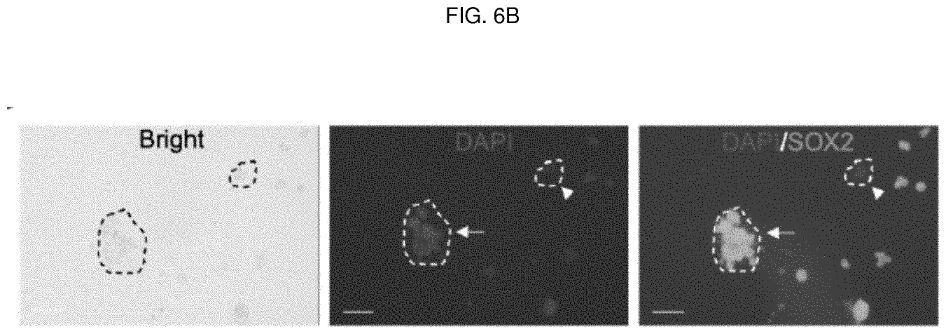

Induction of PKC1 expression post DM (C1) was relatively similar among HepAOs from three donors (FIG. 6A), and organoids were enriched for OCT4 and SOX2 (FIG. 5 and FIG. 6B). The relative expression of hepatic gluconeogenic targets were steady and higher in fresh than frozen and reconstituted organoids. When comparing various EM conditions, conditions with F countered those with A, as expected (FIG. 5A and FIG. 5B), while OSM induction of G6PC and PCK1 expression tend to decrease at one month post differentiation (FIG. 5). FIG. 6 (differentiation of hepatic organoids) shows the fold change in the expression of PKC1 post induction of differentiation in differentiation media (DM) in HepAOs from three donors (FIG. 6A). The difference between donors was not significant. FIG. 6B shows the expression of SOX2 in expanded organoids. Note the nuclear staining of SOX2 in organoids (arrow), while smaller cell structures (arrowhead) didn't show staining. DAPI is a nuclear marker; SOX2 is shown in the third panel in a lighter tone. The scale bar is 100 mm.

### Example 6. Metabolic Competence of 3D Cultured HepGOs

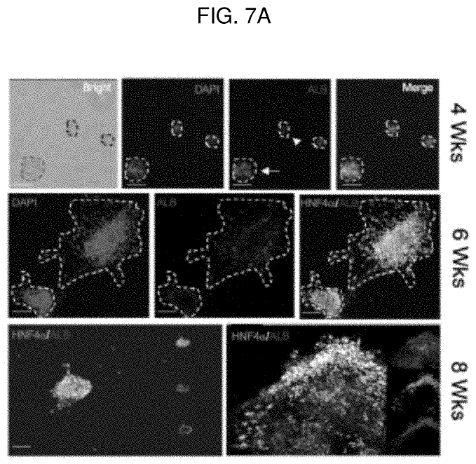

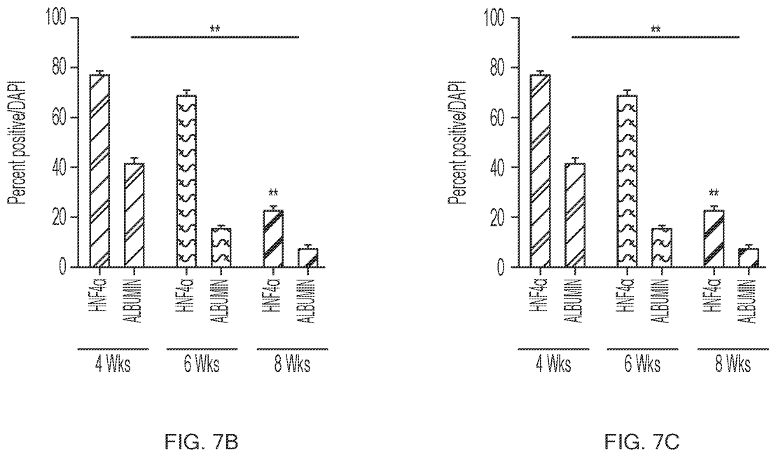

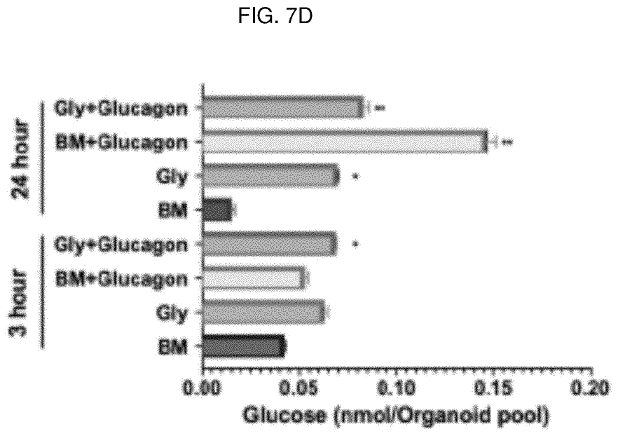

Maintaining expression of functional hepatocytic markers over prolonged culture periods has been a challenge in both 2D and organoid cultures. IF assays were performed to assess the presence of ALB and its continued expression in growing differentiated organoid cultures. FIG. 7, which presents data on HepGOs' dynamic rates of expansion and expression of functional markers.

FIG. 7A presents representative IF images of HepGOs expanded for the indicated times. At 4 weeks, ALB staining in organoids (arrows), while smaller cell structures (arrowhead) showed minimal or no staining. Note the expression of HNF4α (dotted arrows) and the functional marker albumin (ALB) (arrows) over DAPI, the nuclear marker, becomes localized to the periphery of larger organoids in the right side vs. the smaller organoids in left side images of the data for 8 weeks shown in FIG. 7A.

FIG. 7B and FIG. 7C show data on the quantitation of expression of HNF4α (dotted arrows) and the functional marker albumin (ALB) in HepGOs at the indicated times.

FIG. 7D shows data on glucose production in HepGOs expanded in C6 EM supplemented with F+OSM. The HepGOs were starved for 3 or 24 hours and then allowed to produce glucose for 24 hours. The glucose production basal media (BM) was supplemented with either glycerol, glucagon, or a combination of the two. Data was obtained pooled organoids with a total of ˜70,000 organoid cells. The scale bar is 100 μm. (**p<0.01, *p<0.05).

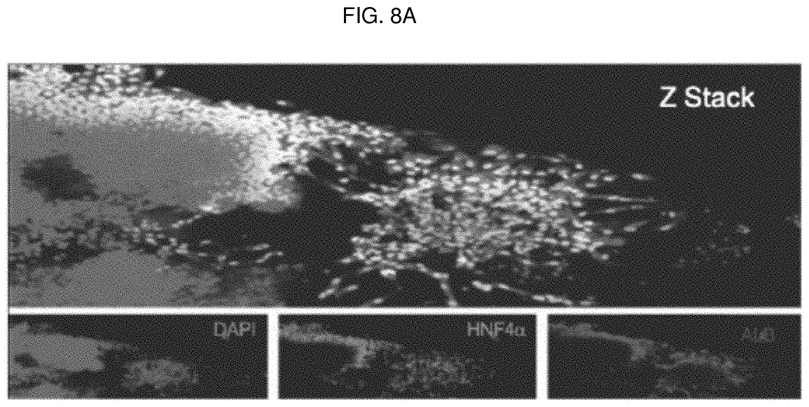

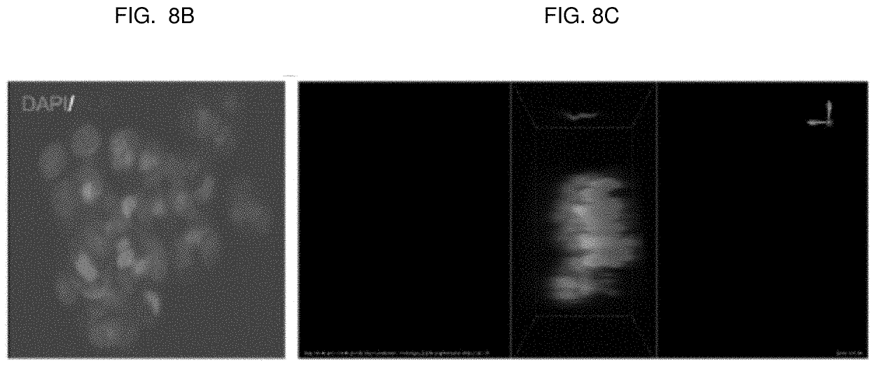

Upon expansion for one month and induction of differentiation for one week, ALB could be detected for an additional 4 weeks (FIG. 7B and FIG. 7C). Notably, at 8 weeks (4-weeks post EM and 3-weeks post induction of differentiation), HepGOs could be distinguished by volumetric analysis into two groups: small organoids (on average 181.43 mm deep×92.50 mm wide) that showed the largest expression of HNF4α/ALB positive cells (see FIG. 7 and FIG. 8), while larger HepGOs showed a central expanding HNF4α/ALB negative core with more HNF4α/ALB positive cells the periphery (FIG. 7B, FIG. 7C and FIG. 8).

FIG. 8 shows localization of nuclear DAPI and cytoplasmic ALB and HNF4α markers in different Z stacks for volumetric analysis of 3D cultured organoids. FIG. 8A is a Z stack confocal Image showing expression of ALB as a functional hepatocyte marker, and HNF4α, a marker used to determine hepatocyte fates. ALB expression is mainly observed around the periphery of the large, differentiated organoids. FIG. 8B shows IF images of a representative organoid used for volumetric analysis. FIG. 8C is a representative volumetric ALB/DAPI overlay for size estimates of organoids. This organoid was determined to approximately measure 181.43 μm deep×92.50 μm wide. The scale bar is 100 mm.

Next, metabolic functions related to gluconeogenesis in these 3D cultured organoids were assessed. The substrate preferences for gluconeogenesis in primary hepatocyte glucose production assays were assessed. In addition, the induction of the rate-limiting enzyme of gluconeogenesis, G6PC upon starvation was demonstrated. Since the 3D organoid culture conditions with EM supplemented with F+OSM generated HepGOs containing cells with hepatocyte-like function, glucose production upon growth factor starvation was assessed in these organoids to determine their metabolic competence. The organoids were serum starved for 3 and 24 hours and allowed to produce glucose for 24 hours. The glucose production media was supplemented with either glycerol, glucagon, or a combination of the two. Glucagon significantly enhanced glucose production in HepGOs after a 24-hour starvation, while glycerol with glucagon had a similar effect in the 3-hour glucose starvation assays (FIG. 7D). These data suggest that HepGOs have metabolic features with cells capable of glucose production in 3D culture upon growth factor starvation.

### REFERENCES

All references listed below and throughout the specification are hereby incorporated by reference in their entirety.

### 1. Chinese Patent Application CN 111004770A.

2. U.S. Pat. No. 10,583,484 to DeMuth et al.
3. Harrison et al., Front. Med. 8(574047): 1-18, 2021.
4. Hindley et al. Dev. Biol. 420″251-261, 2016.

### 5. United States Patent Publication No. 2020-0354683 to Hu et al.

### 6. Kamiya et al., FEBS Lett. 192: 90-94, 2001.

### 7. United States Patent Publication No. 2020-0199538 to Ng et al.

### 8. United States Patent Publication No. 2018-0258400 to Ng et al.

9. International Patent Publication No. WO2017/048193 to Ng et al.

### 10. United States Patent Publication No. 2020-0199537 to Takebe et al.

11. U.S. Pat. No. 8,642,339 to Sato and Clevers.
12. Huch et al., Cell 160: 299-312, 2015; doi: 10.1016/j.cell.2014.11.050.
13. Hu et al., Cell 175: 1591-1606, 2018; e1519, doi: 10.1016/j.cell.2018.11.013.
14. Broutier et al., Nat. Protoc. 11: 1724-1743, 2016; doi: 10.1038/nprot.2016.09710.1038/nprot.2016.097. Epub 2016 Aug. 25.
15. Clevers et al., Cell 165: 1586-1597, 2016: doi: 10.1016/j.cell.2016.05.082 10.1016/j.cell.2016.05.082.
16. Huch et al., Nature 494: 247-250, 2013; doi: 10.1038/nature11826.
17. Peng, W. C.; Logan, C. Y.; Fish, M.; Anbarchian, T.; Aguisanda, F.; Alvarez-Varela, A.; Wu, P.; Jin, Y.; Zhu, J.; Li, B.; et al Inflammatory Cytokine TNFalpha Promotes the Long-Term Expansion of Primary Hepatocytes in 3D Culture. Cell 2018, 175, 745 1607-1619 e1615, doi: 10.1016/j.cell.2018.11.012.
18. Levy, G., Bomze, D., Heinz, S. et al. Long-term culture and expansion of primary human hepatocytes. Nat Biotechnol 33, 1264-1271 (2015). https://doi.org/10.1038/nbt.3377.
19. U.S. Pat. No. 11,034,935 to Huch Ortega.

### Academy of Arts and Sciences (Utrecht, the Netherlands) Huch Ortega M, Clevers J C, Jun. 15, 2021.

20. Wang, B.; Zhao, L.; Fish, M.; Logan, C. Y.; Nusse, R. Self-renewing diploid Axin2(+) cells fuel homeostatic renewal of the liver. Nature 2015, 524, 180-185, doi: 10.1038/nature14863.

1. An isolated hepatocyte in three-dimensional culture, which is derived from a donor adult hepatic cell or a hepatoma, and which is gluconeogenic.

2. A liver organoid comprising at least one hepatocyte of claim 1.

3. A liver organoid of claim 2 which is generated from a single cell of claim 1.

4. A method of producing a liver organoid in serum-free three-dimensional culture conditions, comprising:
(a) obtaining at least one hepatocyte in a single cell suspension;
(b) placing the hepatocyte in three-dimensional organoid culture with extracellular matrix and isolation medium (IM) for 4 days;
(c) substituting the isolation medium with expansion medium (EM) containing about 1 μM to about 10 μM Y-27632 and optionally further containing one or more of about 1 mM to about 20 mM forskolin, about 1 nM to about 1 μM A83-01, about 1 ng/mL to about 20 ng/mL oncostatin M, and about 1 ng/mL to about 50 ng/mL bone morphogenetic protein 7 (BMP7);
(d) culturing the organoid culture for about 1-2 weeks, replenishing the EM about 2-4 times per week;
(e) replacing the EM with differentiation medium (DM);
(f) culturing the organoid culture for about 1 week;
(g) replacing the DM with glucose production medium (GPM) to subject the organoid culture to glucose starvation;
(h) culturing the organoid culture for about 24 hours; and
(i) optionally measuring glucose secretion by the organoid.

5. A method of claim 4, wherein the EM contains about 1 mM to about 20 mM forskolin, about 1 ng/mL to about 10 ng/mL oncostatin M, and about 1 ng/mL to about 50 ng/mL bone morphogenetic protein 7.

6. A method of claim 4, wherein the EM contains about 10 mM forskolin and about 10 ng/mL oncostatin M for the first 6 days of culture in EM and about 10 mM forskolin and about 25 ng/mL BMP7 after 6 days of culture.

7. A method of claim 4, wherein the hepatocytes are adult primary hepatocytes from an adult donor.

8. A method of claim 7, wherein the adult donor is a human.

9. A method of claim 8, wherein single hepatocytes are subjected to a first expansion phase involving 3D culture in EM containing 10 mM forskolin and 10 ng/mL oncostatin M for 6 days and starting at day 7 in a second expansion and differentiation phase involving 3D culture in EM containing 10 mM forskolin and 25 ng/mL BMP7 for 8 days.

10. A method of claim 4, wherein the hepatocytes are obtained from a hepatoma or hepatoma cell line.

11. A method of claim 10, wherein single hepatoma cells are subjected to an expansion and differentiation phase involving 3D culture in EM containing 10 mM forskolin, 10 ng/mL oncostatin M, and 25 ng/mL BMP7 for 8 days.

12. A method of claim 10, wherein the hepatoma or hepatoma cell line show significant reductions in AFP tumor marker expression.

13. A liver organoid produced by the method of claim 4, which is gluconeogenic.
